(19) United States
(12) Patent Application Publication
(10) Pub. No.: US 2015/0174307 A1
(43) Pub. Date: Jun. 25, 2015

(54) CARDIAC ASSIST DEVICE WITH PULSE WAVE ANALYSIS

(71) Applicant: Regents of the University of Minnesota, Minneapolis, MN (US)

(72) Inventors: Peter Eckman, Minneapolis, MN (US); Sameh Hozayen, Trumbull, CT (US); Mohamed Almekkawy, Minneapolis, MN (US); Ashish Singal, Blaine, MN (US)

(21) Appl. No.: 14/415,699

(22) PCT Filed: Jul. 19, 2013
(86) PCT No.: PCT/US2013/051372

§ 371 Date: Jan. 19, 2015

Related U.S. Application Data
(60) Provisional application No. 61/673,684, filed on Jul. 19, 2012.
(60) Provisional application No. 61/730,752, filed on Nov. 28, 2012.

(51) Int. Cl. A61M 1/10

(52) U.S. Cl. CPC A61M 1/1086; A61M 2205/3365

(57) ABSTRACT

A system includes a sensor and a processor. The sensor is configured to generate hemodynamic information for a patient. The processor is configured to execute instructions to calculate spectral content using the hemodynamic information. The processor is configured to generate an output signal based on the calculated spectral content. The calculated spectral content includes a fundamental component and at least one harmonic component. The spectral content corresponds to at least one of amplitude and frequency. The output signal corresponds to a state of the patient or corresponds to an operational parameter of a cardiac assist device associated with the patient.

### CLAIM OF PRIORITY

This patent matter claims the benefit of priority under U.S. Provisional Patent Application Ser. No. 61/673,684 (Attorney Docket No. 600.882PRV), filed on Jul. 19, 2012, and which is hereby incorporated by reference herein in its entirety.

This patent matter claims the benefit of priority under U.S. Provisional Patent Application Ser. No. 61/730,752 (Attorney Docket No. 600.882PV2), filed on Nov. 28, 2012, and which is hereby incorporated by reference herein in its entirety.

### BACKGROUND

Heart failure is a progressive, irreversible disease having a wide prevalence. One treatment approach for a type of heart failure includes an implantable cardiac assist device, sometimes called a ventricular assist device (VAD). One example includes an electrically operated pump having an intake port coupled to the left ventricle and an output port coupled to the aorta. The pump delivers blood at a constant flow rate to the circulatory system. The flow rate is determined by a pump speed. At about the time of implantation, the pump speed can be selected by a physician based on data collected during a procedure commonly referred to as a ramp study.

Patient health can suffer if the blood flow induced by the pump is insufficient or excessive. For example, insufficient blood flow can prevent proper cycling of cardiac valve elements consequently leading to thrombus formation and fusion of leaflets, thus preventing the valve to open and close effectively. Excessive blood flow can lead to hemorrhagic injury. To guard against improper blood flow, a physician may need to adjust pump performance after initial device implantation.

US 20110313238 A1 is entitled FLUID DELIVERY SYSTEM AND METHOD FOR MONITORING FLUID DELIVERY SYSTEM. The abstract states that a fluid delivery system includes an electric motor, a pump driven by the electric motor, and a control system. The control system is programmed to supply a variable voltage to the electric motor, to sense a response of a current of the electric motor to the variable voltage, and to obtain frequency domain information about the response of the current of the electric motor.

US 20060241335 A1 is entitled METHOD AND SYSTEM FOR PHYSIOLOGIC CONTROL OF A BLOOD PUMP. The abstract refers to a physiologic control system and method for controlling a blood pump system such as a VAD system. The pump system includes, for example, a blood pump and a controller for controlling the pump. The system may further include a flow measurement device. Various control schemes are disclosed, including according controlling the pump to achieve one or more of a desired speed, flow rate, or flow pulsatility. Additionally, various methods for determining maximal flow (the maximum flow that can be achieved for the patient while maintaining certain parameters or within certain boundaries) are disclosed.

### OVERVIEW

The present inventors have recognized a problem in selecting a suitable speed for operating a blood pump for a specific patient and in controlling operation of a cardiac assist device. The present subject matter can help provide a solution to this problem, such as by using frequency domain analysis of hemodynamic information to determine a pump speed, for example. A solution can include real-time, or near real-time analysis and control of pump speed. In addition, analysis can identify a failure mode of a cardiac assist device and identify a medical condition or a state of a cardiac valve, for example, of a patient.

An example of the present subject matter includes a processor configured to receive hemodynamic information for a patient. The processor executes instructions to implement an algorithm in which the hemodynamic information (expressed in the time domain) is normalized and transformed to the frequency domain. A power spectrum analysis of the frequency domain can be evaluated using, for example, fundamental amplitude data, harmonic amplitude data, frequency data, ratios of amplitudes, mean values, comparisons, profiles, and other analytics in order to generate an output.

The output can include a signal to indicate a condition of the patient, a condition of the cardiac assist device, or a signal to control an operational parameter of the cardiac assist device. An output signal corresponding to the condition of the patient can indicate a status of a cardiac valve, such as opening and closing of the aortic valve. An output signal corresponding to a condition of the cardiac assist device can indicate that a pump is operating at a true speed that differs from a speed selected by a control signal. An output signal can include a control signal to select a particular speed of a pump of the cardiac assist device.

An example of the present subject matter includes digital signal processing tools and techniques to analyze central pressure signals (from a cardiac assist device) and from peripheral signals (obtained from applanation finger tonometry, sometimes referred to as peripheral arterial tonometry).

These and other examples and features of the present subject matter will be set forth in part in following Detailed Description. This Overview provides non-limiting examples of the present subject matter. This Overview does not provide an exclusive or exhaustive explanation.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A includes a view of a patient with a cardiac assist device and a plurality of sensors, according to one example.

FIG. 1B includes a view of a processor coupled to a sensor, a cardiac assist device, and a communication network, according to one example.

FIG. 1C includes a view of a heart with a cardiac assist device.

FIG. 2 includes a block diagram of a system, according to one example.

FIG. 3 includes a flow chart of a method executed by a processor, according to one example.

FIGS. 4A and 4B include a time domain view and a frequency domain view, respectively, of hemodynamic information for a particular patient with a pump operating at a first speed, according to one example.

FIGS. 5A and 5B include a time domain view and a frequency domain view, respectively, of hemodynamic information for a particular patient with a pump operating at a second speed, according to one example.

FIGS. 6A and 6B include a time domain view and a frequency domain view, respectively, of hemodynamic information for a particular patient with a pump operating at a third speed, according to one example.

FIGS. 7A and 7B include a time domain view and a frequency domain view, respectively, of hemodynamic information for a particular patient with a pump operating at a fourth speed, according to one example.

FIGS. 8A and 8B include a time domain view and a frequency domain view, respectively, of hemodynamic information for a particular patient with a pump operating at a fifth speed, according to one example.

FIG. 9 includes a three-dimensional plot in the frequency domain at selected pump speeds, according to one example.

FIG. 10A includes a bar graph of mean value of power spectrum for selected pump speeds, according to one example.

FIG. 10B includes a bar graph of RMS value of power spectrum for selected pump speeds, according to one example.

FIGS. 11A, 11B, and 11C illustrate signal amplitude at selected pump speeds, for a fundamental frequency, a first harmonic, and a second harmonic, respectively.

FIGS. 12A, 12B, and 12C illustrate ratios of signal amplitudes at selected pump speeds.

FIG. 13 includes a flow chart of a method executed by a processor, according to one example.

### DETAILED DESCRIPTION

Part 1 includes a structural description; Part 2 includes a description of methods; Part 3 includes a description of various examples; and Part 4 includes additional notes.

### Part 1

### Structure

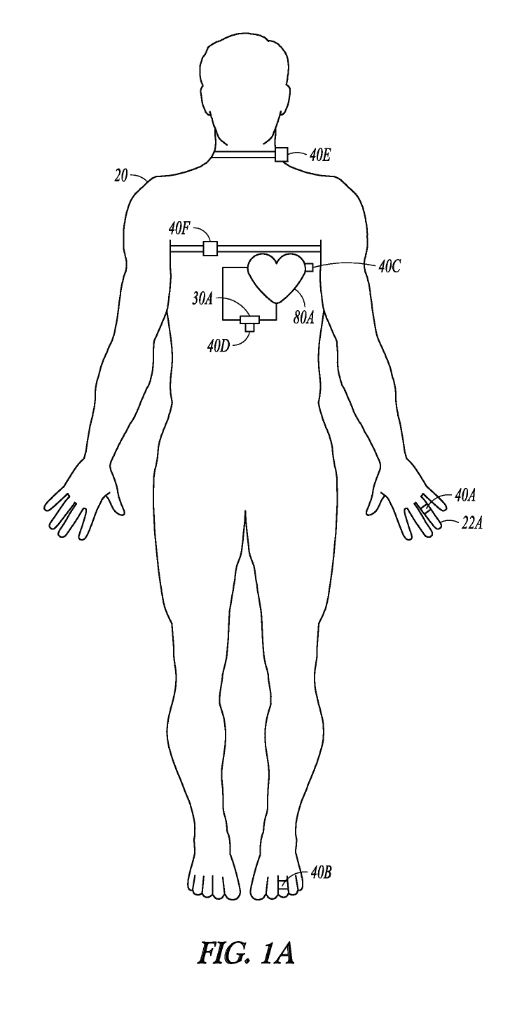

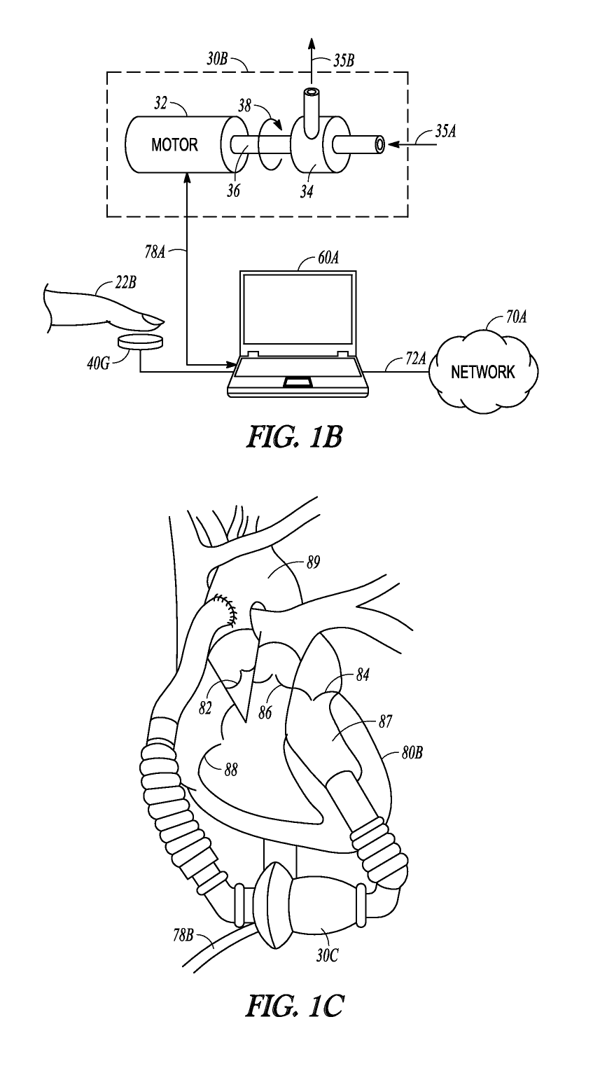

FIG. 1A includes a view of patient 20 with cardiac assist device 30A coupled to heart 80A.

Cardiac assist device 30A can include an implanted device, an external device, or a hybrid device having both an implanted element and an external element. Cardiac assist device 30A can be, for example, a VAD. A ventricular assist device is a mechanical circulatory device configured to aid the heart in pumping blood at a sufficient rate to meet the metabolic demands of the body. A VAD, sometimes referred to as a heart pump or pump, can partially or completely take over the function of a failing heart. Cardiac assist device 30A can include a right ventricular assist device (RVAD), a left ventricular assist device (LVAD), a bi-ventricular assist device (BiVAD), or a total artificial heart (TAH).

One example of a cardiac assist device 30A includes a cuff or balloon that is pneumatically inflated and deflated in order to modulate pressure in a chamber of heart 80A and thereby pump blood.

The figure illustrates a plurality of sensors in relation to patient 20. In one example of the present subject matter, hemodynamic information provided by at least one sensor is accessed and processed in accordance with a method described herein. A sensor can be invasive (implantable) or non-invasive (external).

Sensor 40A is an example of a non-invasive sensor coupled to finger 22A of patient 20. Sensor 40A can include a peripheral applanation tonometry sensor. A tonometer provides a signal associated with displacement, such as a change in volume or a change in pressure. A tonometry sensor can include a piezoelectric element, an optical emitter element and an optical detector element, a capacitance-sensing element, or other type of sensor. Sensor 40A can include a cuff, a touch-pad, or other contact surface.

Sensor 40A is configured to detect hemodynamic information and generate an electrical output signal, in a continuous time series. The output signal from sensor 40A is a time domain representation of the hemodynamic information. The hemodynamic information can include a pulse, a temperature, blood oxygenation, or other time varying physiological data. Sensor 40A can be coupled to any peripheral limb.

Sensor 40B, in the example shown, includes a sensor configured to detect hemodynamic information associated with a toe of patient 20. Both sensor 40A and sensor 40B can include a PAT sensor element.

Sensor 40C is configured to detect hemodynamic information at heart 80A. Sensor 40C can include an implantable sensor (such as a catheter-based balloon-type sensor) or an external sensor (such as an acoustical sensor) tailored to provide information as to physiological information of heart 80A. In one example, this can include a sensor configured to detect a pulse, a pressure, a flow at an aortic anastomosis port.

Sensor 40D is configured to provide a time domain signal associated with cardiac assist device 30A. Data provided by sensor 40D can include, for example, information as to operation of a pump. The data can represent an electric current draw associated with an electric motor, an electrical resistance or impedance, a fluid flow rate, or a temperature. In addition, sensor 40D can be configured to provide data as to a pulse, a pressure, or a flow rate, measured at a blood intake port of a pump or at a blood output port of a pump.

Sensor 40E can be configured to generate hemodynamic information detected at a site associated with an artery. In the figure, sensor 40E is configured to provide an output corresponding to a carotid artery of patient 20. Other arteries are also contemplated such as the radial artery. Sensor 40F can be configured to generate hemodynamic information detected at an external site, here shown as the chest of patient 20. Sensor 40F is retained in position by a chest strap.

An electrical signal generated by (or provided by) any one or more of sensors 40A-40F is provided to a processor (not shown in this view) for analysis. For instance, one example of the present subject matter can include sensor 40A and another example can include sensor 40D and sensor 40E.

FIG. 1B illustrates a view of processor 60A coupled to sensor 40G, cardiac assist device 30B, and communication network 70A, according to one example.

Processor 60A, in the example illustrated, includes a computer. The figure illustrates processor 60A in the form of a laptop or portable computer. Processor 60A can include an analog-to-digital converter (ADC), a filter, an amplifier, or other circuitry configured to receive a time domain signal and generate a digital signal. Processor 60A is configured to execute a set of instructions (stored in a memory) to implement an algorithm and perform a method described elsewhere in this document. In the example shown, processor 60A includes a keyboard or other user input device to allow a user to indicate a selection or provide an instruction. Processor 60A includes a graphical display to provide visual data to a user.

Sensor 40G, in the example illustrated, includes a piezoelectric element configured to detect applanation of a phalange, here depicted as finger 22B of patient 20. Sensor 40G is coupled by a wired connection or a wireless channel to processor 60A.

Cardiac assist device 30B, in the example illustrated, includes electric motor 32 coupled by shaft 36 to blood pump 34. Blood pump 34 has a blood intake port to receive blood flowing in the direction shown at arrow 35A and a blood output port to deliver blood flowing in the direction shown at arrow 35B. Rotation of shaft 36, in the direction shown at arrow 38, produces blood flow through pump 34 as shown by arrows 35A and 35B. Pump flow and pressure are determined, at least in part, by the speed of rotation of shaft 36.

Cardiac assist device 30B can be a shaft-less pump having, for example, a plurality of electrically controllable windings arranged about a fluid chamber. In such an example, pump rotation is not measured at a physical shaft but instead, can be viewed as the speed of sequential actuation of the windings.

Cardiac assist device 30B is coupled to processor 60A by link 78A. Link 78A can include a wired connection or a wireless channel and can communicate a signal in an analog format or in a digital format.

Link 78A can include a wired connection or a wireless channel, either of which can be configured for unidirectional or bidirectional communication. For example, processor 60A can be configured to provide a unidirectional control signal on link 78A to select a rotation speed of cardiac assist device 30B. In one example, cardiac assist device 30B is configured to provide a unidirectional signal on link 78A to processor 60A in which the signal corresponds to a sensed electrical parameter or physiological parameter associated with patient 20. In one example, link 78A is configured as a bidirectional signal in which a measured or detected signal from cardiac assist device 30B is communicated to processor 60A and in which processor 60A provides a control signal to cardiac assist device 30B.

Processor 60A is coupled to network 70A by link 72A. Network 70A can include a local network (such as a local area network or LAN), or a wide area network (such as the internet). In various examples, network 70A is in communication with a physician or other care provider or in communication with a patient.

Link 72A can include a wired connection or a wireless channel and can communicate a signal in an analog format or in a digital format. Link 72A can be unidirectional or bidirectional.

FIG. 1C includes a view of heart 80B with cardiac assist device 30C. Cardiac assist device 30C has an intake port coupled to heart chamber 87 (here shown as a left ventricle) and an output port coupled to vessel 89 (here shown as the aorta). Cardiac assist device 30C is coupled by link 78B to a processor (not shown in this view). Cardiac assist device 30C can take other forms or be coupled to other chambers or vessels.

Heart 80B includes mitral valve 84, tricuspid valve 88, aortic valve 82, and pulmonary valve 86. As explained elsewhere in this document, the state of a particular cardiac valve, such as aortic valve 82, can be determined based on analysis of hemodynamic information.

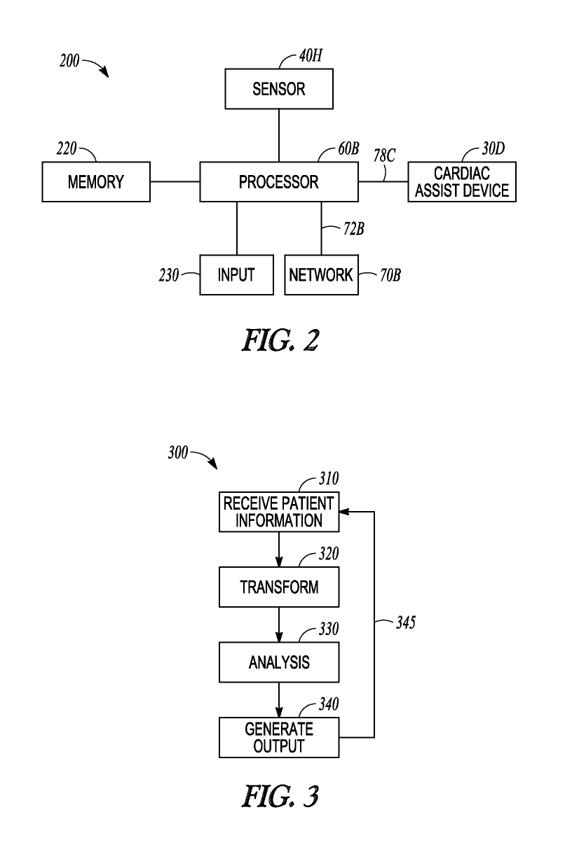

FIG. 2 includes a block diagram of system 200, according to one example. In the figure, processor 60B is coupled to sensor 40H, memory 220, input 230, network 70B, and cardiac assist device 30D.

Processor 60B can include a digital computer. For example, processor 60B can include a microprocessor along with ADC circuitry, a filter, an amplifier, a mixer, or other module. In one example, processor 60B includes an analog signal processor. Processor 60B can be configured to generate data for use in determining a suitable pump speed.

Sensor 40H can include a peripheral sensor configured to generate an electrical signal corresponding to hemodynamic information. In other examples, sensor 40H provides a signal correlated with operation of a cardiac assist device personal to the patient.

Memory 220 can include digital or analog memory for use by processor 60B. Memory 220 can provide storage for reference data, such as historical data for a particular patient or for a population of patients. In addition, memory 220 can provide storage for signature or trend information. Memory 220 can provide storage for instructions executable by processor 60B.

Input 230 can include a keyboard, a cursor control device (mouse or trackball), a touch-screen, a microphone, or other input device for controlling operation of processor 60B. In various examples, input 230 is operable by a patient or by a physician or care provider.

Network 70B can include a narrow or wide area network configured to notify a patient or a physician (or care provider) with operational or diagnostic information. In addition, instructions for operation of processor 60B can be received using network 70B and link 72B.

Cardiac assist device 30D is coupled to processor 60B by link 78C.

System 200 can be fabricated in one or more housing modules. For example, a unitary implantable module can include processor 60B, memory 220, cardiac assist device 30D, and sensor 40H. In this example, input 230 and network 70B are coupled by wireless links to processor 60B. As another example, cardiac assist device 30D is fully implanted and sensor 40H, processor 60B, and memory 220 share a common housing and are connected by a wired or wireless connection with input 230 and network 70B.

### Part 2

### Methods

FIG. 3 includes a flow chart of method 300 executed by a processor, according to one example. At 310, method 300 includes receiving patient information. The patient information can include hemodynamic information provided by a sensor, such as sensor 40A (FIG. 1A). Hemodynamic information can include a pulse signal, a flow rate, a temperature or other data. Patient information can include data as to performance of a cardiac assist device associated with the patient. For example, with an electrically operated cardiac assist device, an electrical current or an electrical resistance can provide information as to the patient. In a similar manner, a pneumatic pressure or flow rate associated with operation of an inflation-based cardiac assist device can provide patient information.

At 320, the patient information is transformed from a time domain representation to a frequency domain representation. This can include normalizing an amplitude or normalizing a frequency component, conducting a Fourier transform to generate spectral content, and calculating a power spectrum representative of the patient information. In one example, processor 60A (FIG. 1B) executes instructions to calculate the transform.

At 330, the frequency domain representation is analyzed. Analysis can include comparing calculated data with reference data. The calculated data can include at least one of an amplitude, a frequency, a phase, a trend, a mean value, a standard deviation, a value generated by a mathematical operation, or a statistical calculation, any of which can be viewed as a statistical profile. The reference data can be derived from the patient, derived from a model, derived from a population, or derived from another source. Analysis can include evaluating a trend in the calculated data. For example, analysis can include calculating a ratio based on the spectral content or a mean (or a root mean square value). In one example, processor 60A (FIG. 1B) executes instructions to conduct the analysis. Over a period, the spectral content can change in amplitude, frequency, or both amplitude and frequency.

At 340, an output is generated based on the outcome of the analysis. The output can include a signal to select a pump speed (for a cardiac assist device), a signal to notify a user of a condition or operational parameter associated with a cardiac assist device (for example, excessive electrical current draw), or notify a user of a medical diagnosis associated with the patient. In one example, processor 60A (FIG. 1B) executes instructions to generate the output.

After generating the output at 340, processing returns to again receive patient information (at 310), as indicated by path 345. In this iterative manner, real-time, or near real-time, pump speed adjustments can be determined.

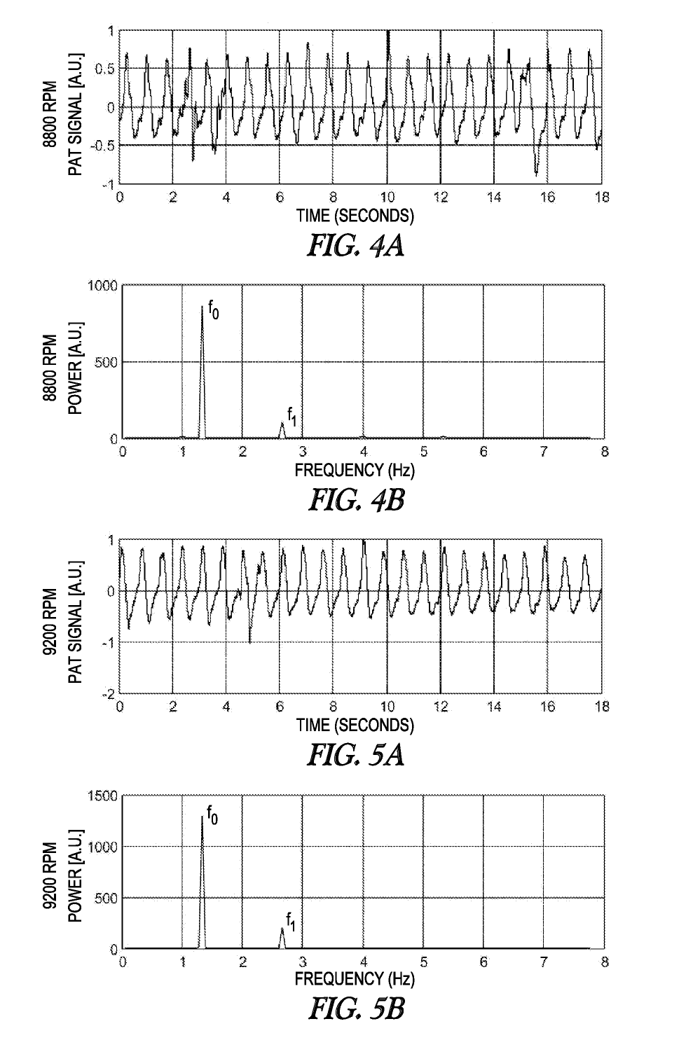

FIGS. 4A-8A and FIGS. 4B-8B illustrate time domain and frequency domain, respectively, representations of hemodynamic information corresponding to a patient having a cardiac assist device operating at selected pump speeds.

Each of FIGS. 4A-8A illustrates an example of a time domain representation of a pulsatile signal as generated, for example, by a peripheral applanation tonometry sensor. In FIGS. 4A, 5A, 6A, 7A, and 8A, the pump speed is 8,800 revolutions per minute (RPM), 9,200 RPM, 10,000 RPM, 11,200 RPM, and 11,600 RPM, respectively. In each of the figures, the abscissa indicates a window of 18 seconds and the ordinate indicates arbitrary units of amplitude with excursions in both positive and negative directions. The amplitude shown in FIGS. 4A-8A is normalized.

Each of FIGS. 4B-8B illustrates an example of a frequency domain representation of the hemodynamic information depicted in FIGS. 4A-8A, respectively, and calculated by a processor, such as processor 60B (FIG. 2). In each of the figures, the abscissa is calibrated in units of Hertz (Hz) and the ordinate is calibrated in arbitrary units of power (and displayed on different scales).

In FIG. 4A, at a pump speed of 8,800 RPM, the pulsatile nature of the signal is apparent from the periodic pattern that, in this example, corresponds to a pulse rate of approximately 90 beats per minute. FIG. 4B indicates notable spectral content at frequency f₀, sometimes referred to as the fundamental frequency, and at frequency f₁, sometimes referred to as the first harmonic. As shown, frequency f₀ is approximately 1.3 Hz and has an amplitude of approximately 900 arbitrary units and frequency f₁ is approximately 2.6 Hz and has an amplitude of approximately 100 arbitrary units. A pulse rate of 60 beats per minute has a fundamental frequency f₀ of 1 Hz, a first harmonic f₁ of 2 Hz, and a second harmonic f₂ of 4 Hz.

FIG. 5A, at a pump speed of 9,200 RPM, also exhibits the pulsatile nature of the signal. Relative to the content shown in FIG. 4B, the data shown in FIG. 5B indicates an increase in fundamental frequency (from approximately 800 in FIG. 4B, to approximately 1200 in FIG. 5B) and an increase in amplitude of the first harmonic (from approximately 70 in FIG. 4B, to approximately 200 in FIG. 5B).

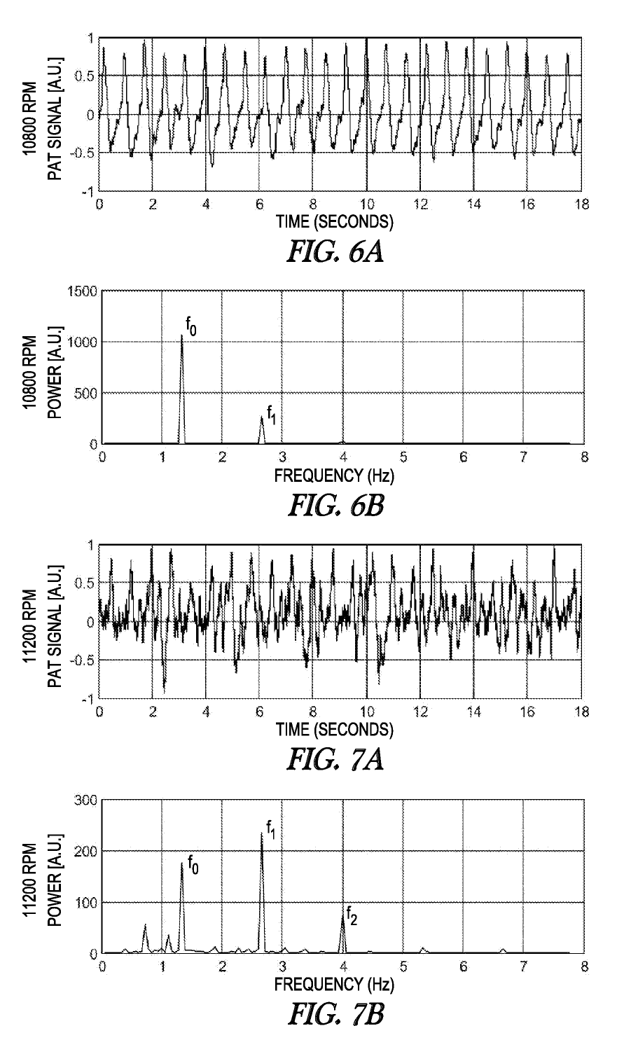

FIG. 6A, at a pump speed of 10,000 RPM, also exhibits the pulsatile nature of the signal. FIG. 6B indicates a slight reduction in amplitude of the fundamental frequency and another slight increase in amplitude of the first harmonic.

FIG. 7A, at a pump speed of 11,200 RPM, exhibits signs of both rapid and slow fluctuations in the pulsatile signal. FIG. 7B indicates notable low frequency content (below fundamental frequency f₀), high frequency content at, for example, 4 Hz, a slight amplitude reduction and downward shift of the fundamental frequency, and a substantial increase in amplitude of the first harmonic.

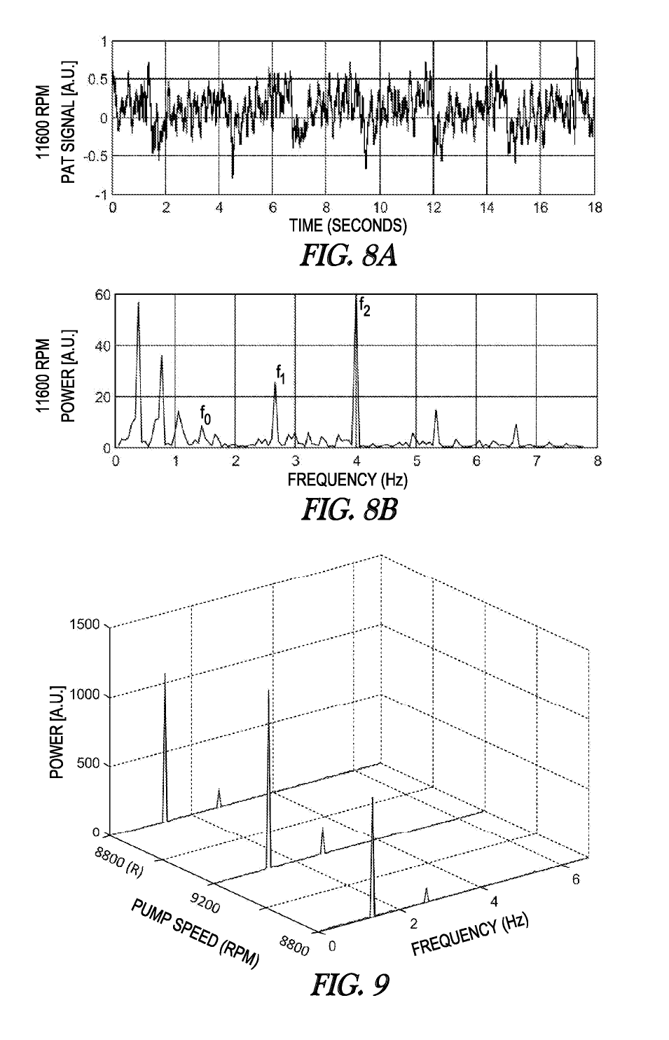

FIG. 8A, at a pump speed of 11,600 RPM, also exhibits signs of both rapid and slow fluctuations in the pulsatile signal. FIG. 8B indicates substantial low frequency content and increased high frequency components, at for example, 4 Hz.

FIG. 9 illustrates a three-dimensional plot of power spectra (in the frequency domain) at selected pump speeds, according to one example. The data shown in FIG. 9 is derived from the example data shown in FIGS. 4B-8B. The frequency axis is calibrated in Hz and illustrates the spectral content of the power spectrum. The amplitude of each spectral component is represented in arbitrary units of power on the vertical axis. The figure depicts relative values of amplitude for pump speeds of 8,800 RPM and 9,200 RPM.

As shown in the figure, the state of the aortic valve is correlated with the pump speed. For example, at a first instance of operation at a speed of 8,800 RPM (foreground row of data), the aortic valve is open. When the pump is operated at 9,200 RPM (middle row of data), the aortic valve is closed. When the pump is again operated at 8,800 RPM (background row of data), indicated by suffix “R” in the illustrated plot, the aortic valve returns to an open state. Stated differently, a pump speed of 8,800 RPM can be viewed as the baseline for which the aortic valve is open. A change in the power spectral signature is correlated with a change in the pump speed.

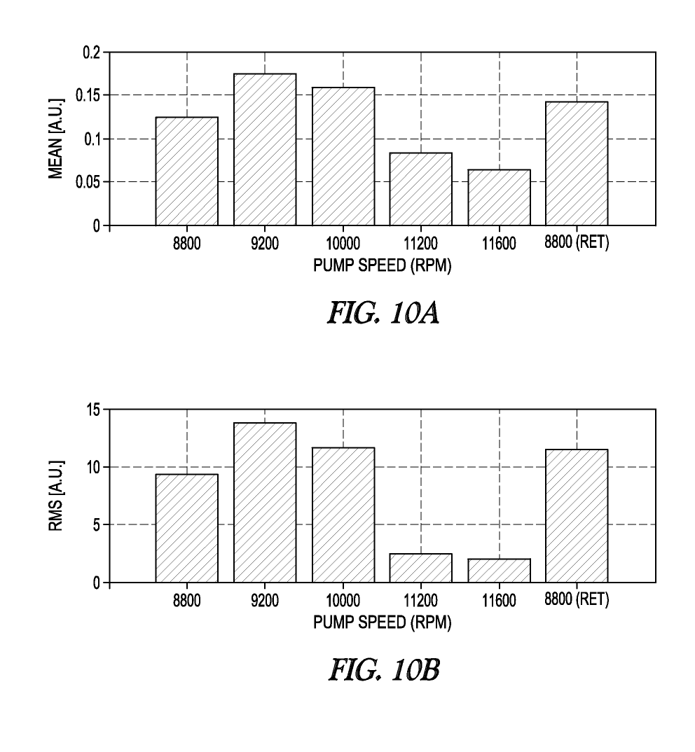

The valve actuation correlates with a mean value of the power spectrum as shown in FIG. 10A (mean) and FIG. 10B (root mean square, or RMS). In both FIGS. 10A and 10B, the mean value and the RMS value of the power spectrum (shown in arbitrary units) exhibits a peak value at 9,200 RPM. According to one example, the valve state can be determined by calculating a percentage change in difference of in mean value or in RMS value.

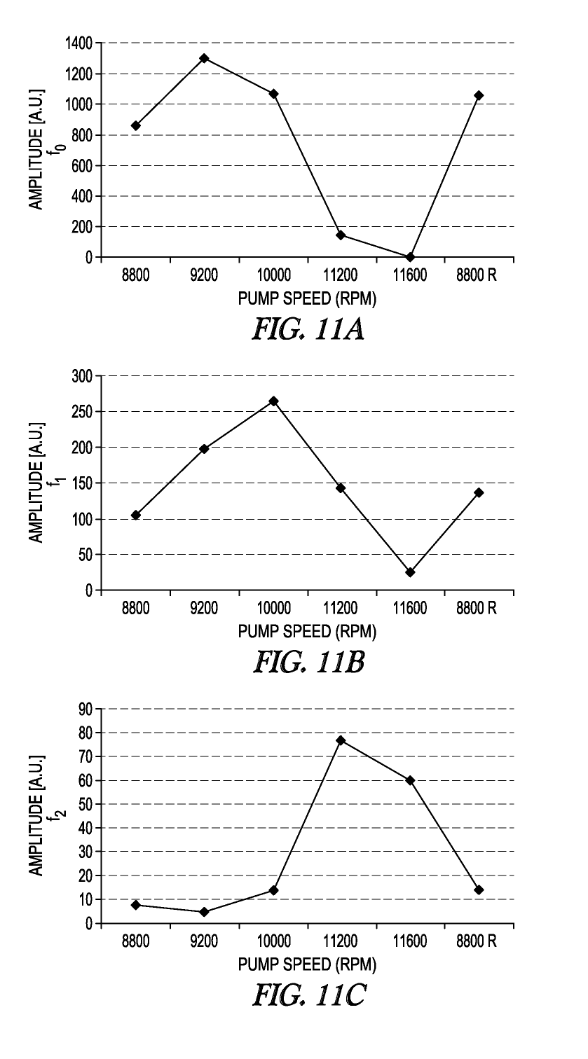

FIGS. 11A, 11B, and 11C illustrate changes in the signal amplitude at selected pump speeds, for a fundamental frequency, a first harmonic, and a second harmonic, respectively. The data illustrates a correlation between the spectral content of the hemodynamic information and the operational parameter of the cardiac assist device (in this example, the pump speed is construed as the operational parameter).

As shown in FIG. 11A, with increasing pump speed, the amplitude of the fundamental frequency exhibits an increase as speed rises to 9,200 RPM followed by a drop in amplitude as the speed continues to rise to 11,600 RPM. Coincident with a return to 8,800 RPM, (denoted with suffix “R”) the amplitude of the fundamental frequency returns to approximately the same value as noted pre-cycle. FIG. 11B shows a peak amplitude in the first harmonic at a pump speed of approximately 10,000 RPM and FIG. 11C shows a peak amplitude in the second harmonic at a pump speed of approximately 11,200 RPM. The pump speed can be determined by amplitude of the fundamental frequency or a harmonic.

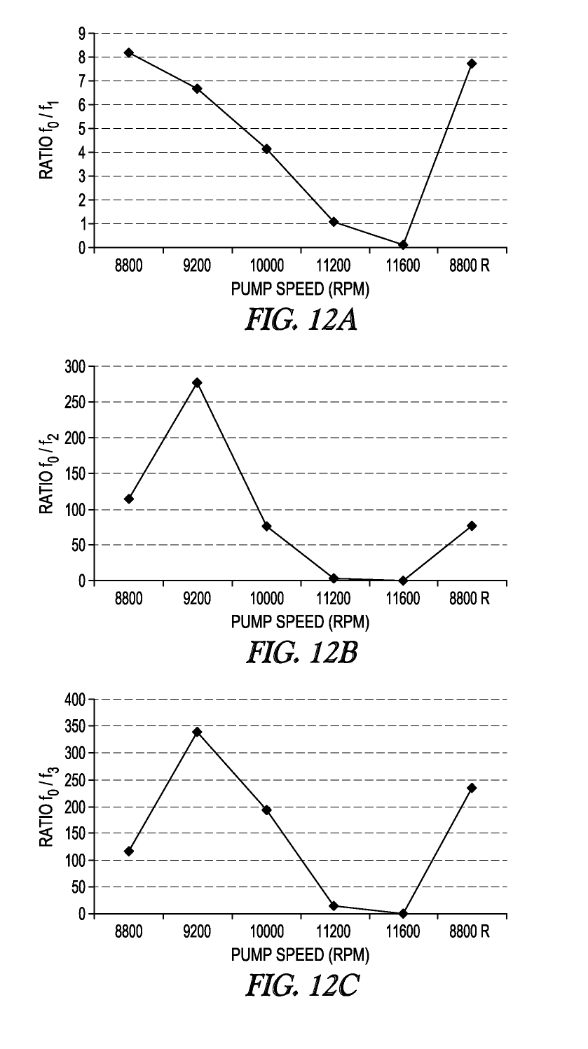

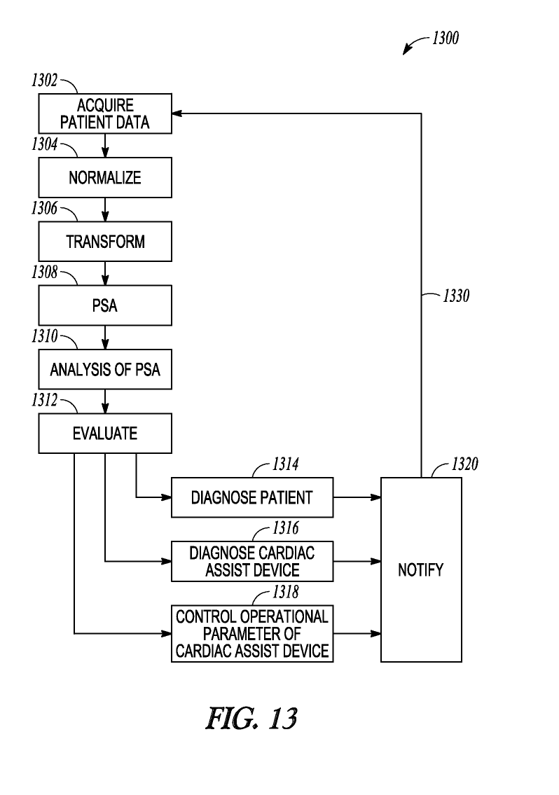

Ratiometric analysis of power spectrum amplitudes provides a normalized view that tends to attenuate sensitivity to variations in raw numerical values. FIGS. 12A, 12B, and 12C illustrate examples of ratios calculated from the fundamental and harmonic frequencies. The calculated ratios can be used to determine the state of a cardiac valve.

FIG. 12A illustrates the changes in the ratio of f₀/f₁ as a function of pump speed. In the example shown, at a pump speed of 8,800 RPM, the aortic valve is open and ratio f₀/f₁ has a value corresponding to approximately 8 and, at a pump speed of 9,200 RPM, the aortic valve is closed and ratio f₀/f₁ has a value corresponding to approximately 6.5.

FIG. 12B illustrates f₀/f₂ as a function of pump speed. In the example shown, at a pump speed of 8,800 RPM, the aortic valve is open and ratio f₀/f₁ has a value corresponding to approximately 120 and, at a pump speed of 9,200 RPM, the aortic valve is closed and ratio f₀/f₂ has a value corresponding to approximately 275. When the pump speed is returned to 8,800 RPM, the aortic valve returns to an open position and the ratio f₀/f₁ has a value corresponding to approximately 75.

FIG. 12C illustrates f₀/f₃ as a function of pump speed. In the example shown, at a pump speed of 8,800 RPM, the aortic valve is open and ratio f₀/f₃ has a value corresponding to approximately 120 and, at a pump speed of 9,200 RPM, the aortic valve is closed and ratio f₀/f₃ has a value corresponding to approximately 340. When the pump speed is returned to 8,800 RPM, the aortic valve returns to an open position and the ratio f₀/f₃ has a value corresponding to approximately 240.

Other ratios can also be calculated using the spectral content and correlated to pump speed, correlated to valve state, correlated to patient state and patient health.

FIG. 13 includes a flow chart of method 1300 executed by a processor, according to one example.

At 1302, method 1300 includes acquiring patient data. Patient data can include hemodynamic information as well as information concerning a cardiac assist device associated with a particular patient. This can include an electrical current draw, a resistance or impedance measurement, a voltage, a power, a frequency, or other measured data corresponding to operation or state of the cardiac assist device.

At 1304, method 1300 includes normalizing the time domain data. The patient data can be normalized based on amplitude or based on frequency.

At 1306, method 1300 includes performing a transform. The transform, in one example, includes conducting a fast Fourier transform (FFT). Other transforms are also suitable, including a Green's function, a Laplace transform, or a Z-transform. The transform function resolves the time domain data into a frequency domain representation having complex spectral content including a combination of amplitude, phase, or frequency.

At 1308, method 1300 includes calculating a power spectrum. This can include executing a squaring function that yields a positive and real power per frequency representation. The power spectrum can be expressed in decibels as a function of frequency.

At 1310, method 1300 includes analyzing the power spectrum. Analysis can include calculating a frequency, an amplitude, a ratio harmonics, a product of harmonics, a sum of harmonics, or a difference of harmonics. The operators (division, multiplication, difference, and addition) can be combined or applied repeatedly in order to derive a measure correlated with the health of the patient, condition of the cardiac assist device, or in order to determine a value for a control signal for the cardiac assist device. The control signal is configured to select a particular value for at least one operational parameter of the cardiac assist device.

At 1312, method 1300 includes evaluating the analyzed power spectrum in order to generate an output based on a comparison with reference data or based on analysis (without comparison with a reference). Evaluating can also include executing an algorithm to implement an artificial intelligence tool, a neural network, or other learning function.

Following evaluating at 1312, method 1300 can be tailored to suit a particular objective. FIG. 13 illustrates an example including three options; however, a particular embodiment can have any one, any two, or all three options implemented. For example, method 1300 can include, at 1314, generating a patient diagnosis. The patient diagnosis can include an output that identifies the state of a cardiac valve such as an aortic valve, a mitral valve, a pulmonary valve, or a tricuspid valve. The valve state can be fully open, fully closed, partially open, partially closed, fluttering, prolapsed, or any other state.

The patient diagnosis can include diagnosing a medical condition such as coronary artery disease (CAD), ischemic heart disease, hypertension, congestive heart failure (CHF), left-sided heart failure, right-sided heart failure, bi-ventricular heart failure, systolic heart failure (SHF), diastolic heart failure (DHF), systolic dysfunction, diastolic dysfunction, acute decompensation, aortic insufficiency (AI), aortic regurgitation (AR), aortic stenosis (AS), mitral regurgitation (MR), mitral insufficiency (MI), mitral stenosis (MS), and a cardiac valvular disease, or any other form of cardiomyopathic disease. These and other medical conditions can be diagnosed using an artificial intelligence, neural network, or learning algorithm.

In one example, method 1300 can include, at 1316, diagnosing a condition of the cardiac assist device. For example, a fault condition is indicated if the control signal provided to the pump specifies that the pump is to operate at a particular speed and the analyzed power spectrum is inconsistent with that particular speed.

In one example, method 1300 can include, at 1318, generating an output signal tailored to select an operational parameter of the cardiac assist device. For example, the control signal can call for the cardiac assist device to operate a pump at a particular speed, change a duty cycle for operating a pump, change the rate of acceleration of a pump speed, change a pressure or temperature, or make other changes in the operational parameters of a cardiac assist device. More than one operational parameter can be modulated using one or more output signals from processor 60B.

In various examples, multiple objectives (such as that at 1314, 1316, and 1318) are achieved in a parallel or sequential manner.

At 1320, method 1300 includes notification. Notification can include communicating an alert message to the patient, communicating an alert message to a physician, or communicating a message to a patient to announce a change in an operational parameter of the cardiac assist device. In one example, notification includes storing data in memory 220 or communicating data using network 70B (FIG. 2).

Method 1300, in the example illustrated, includes pathway 1330 by which processing returns to acquiring patient data after notification. Pathway 1330 provides a feedback route by which updated patient data is acquired, updated analytics are calculated, and an updated output is generated. Pathway 1330 allows continuous or iterative data analysis and adjustment on a real-time basis or on a near real-time basis. A near real-time basis can include conducting calculations at a repetition rate of several times per hour. Method 1300 can be configured to automatically adjust an operational parameter of a cardiac assist device in order to achieve a particular performance objective. The performance objective can be optimization of valve operation, power consumption, blood pressure level, circulation rate, or other parameter.

Variations of method 1300 are also contemplated. For example, pathway 1330 can be omitted and, in this instance, following evaluation (at 1312), the patient diagnosis (at 1314) can be communicated (notify, at 1312) to a physician or to the patient. In this example, the patient diagnosis can lead to a change in therapy or adjustment of an operational parameter of the cardiac assist device.

### Part 3

### Examples

An example of the present subject matter can be configured to learn, and store in memory 220, certain aspects of the spectral content of a patient. The spectral content can be correlated with a state of the patient. The patient state can include, for example, supine, sitting standing, walking, and running. With reference to FIG. 2, the patient state can be communicated to processor 60B by way of memory 220, input 230, or network 70B. Memory 220 can store patient state information and processor 60B can correlate the patient state information with the spectral content.

In one example, processor 60B is configured to execute a method (such as method 1300 shown in FIG. 13) to adjust an operational parameter for cardiac assist device 30D in order to achieve a predetermined spectral content.

In one example, as the spectral content changes over time, processor 60B identifies and stores (in memory 220) archival data corresponding to trends and variations in order to more precisely match the spectral content. Memory 220 can store sensor information from a sensor (such as sensor 40A-40H), transform data and calculations, analysis data and intermediate calculations, spectral content, and graphical and numerical results. The stored data can be used for comparisons or other analysis.

Spectral analysis of the hemodynamic information can be used to identify changes in the operation of a cardiac assist device. For example, when a heart pump is turned off, the pulsatile index (as detected by sensor 40A) will appear as a change in the frequency domain.

One example of the present subject matter is configured to generate a feedback signal to control operation of a cardiac assist device. In this example, a physiological signal derived from a non-invasive applanation tonometry sensor and an electrocardiograph signal derived from a surface electrode is provided to processor 60B. Processor 60B is configured to generate a frequency domain spectrum from the time domain signals and configured to conduct digital signal processing. The signal processing can include, for example, generating a fast Fourier transform, principal component analysis, or Fourier series analysis. Signal processing can also include conducting peak ratio analysis or peak separation analysis. Processor 60B is configured to generate an output based on the signal processing. The output can include a clinical diagnosis or a biofeedback signal. The biofeedback signal can be communicated to the cardiac assist device to adjust an operational parameter. In one example, patient information from an electrocardiograph signal is processed and analyzed in accordance with the methods described herein.

In one example, processor 60B is configured to generate an output based on a signal derived from a cardiac assist device. The signal from the cardiac assist device can include a time domain representation of electrical power consumption. Digital signal processing can be used to generate a feedback signal for controlling an operational parameter of the cardiac assist device.

In one example, processor 60B is configured to compare operational performance of a cardiac assist device with a predetermined matched state. The output generated by processor 60B is communicated to cardiac assist device 30D and is configured to reduce or minimize the error between the two states.

In one example, an output is generated based on digital signal processing including filtering and analysis. Signal processing can include performing a Fourier transform, power spectral analysis, and principal component analysis. One example includes executing a Volterra filtering function. A Volterra filter can be used with applanation tonometry signals to perform power spectral analysis of fundamental, harmonic, and ratiometric frequency components. A second order Volterra filter can be implemented using adaptive filtering coefficients and a least mean square (LMS) algorithm. The frequency spread and power spectral intensity of fundamental, first and second harmonics can be calculated to identify vascular function and identify differential signatures as between a reference signal and various disease states.

In one example, processor 60B is configured to generate an output based on hemodynamic information from a first sensor (such as sensor 40A, FIG. 1A) and based on kinematic information from a second sensor (such as sensor 40F or sensor 40D, FIG. 1A). Kinematic information can include information regarding acceleration, posture, or position of patient 20. Processor 60B is configured to receive the sensor signals and generate an output based, in part, on a posture of the patient. One example includes a position sensor responsive to 3-axes and is coupled to, or built into, cardiac assist device 30D. Processor 60B generates an output corresponding to an operational parameter of cardiac assist device 30D and tailored to the position of patient 20.

One example is configured to adjust an operational parameter of cardiac assist device 30D based on metabolic demands of patient 20. Light to moderate physical exertion can increase blood flow demands on cardiac assist device 30D as compared to when the patient is resting. Real-time hemodynamic information as to heart rate and rhythm is generated by an electrical sensing circuit built into cardiac assist device 30D. Processor 60B generates an output corresponding to an operational parameter of cardiac assist device 30D and tailored to meet the rate demands of patient 20. The operational parameter can relate to blood flow or electrical power or other parameter.

Cardiac assist device 30D performance can be adjusted or optimized using the output signal from processor 60B. The output signal can be viewed as a biofeedback signal configured to achieve a particular objective based on a detected patient condition.

An example is configured to detect cardiac valve opening, valve closing, or both valve opening and closing. A medical risk of LVAD implant is aortic valve regurgitation associated with infrequent opening of the aortic valve that can lead to commissural fusion, insufficient central coaptation, further leading to aortic insufficiency. Significant aortic regurgitation can lead to an effective fistula for blood flow and compromise effective cardiac output. One example of the present subject matter includes periodic evaluation of a valve state in order to mitigate this risk. The valve can be the aortic valve, the mitral valve, the pulmonary valve, or the tricuspid valve.

One example is configured to detect cardiac arrhythmia. In this example, sensor 40H is configured to generate a signal corresponding to electrical activity from the myocardium. Sensor 40H can include on internal electrode, an external electrode, or can be configured to detect a change in applanation tonometry. In one example, the output from processor 60B is configured to modulate an operational parameter of cardiac device 30D in order to increase blood flow to attempt mechanical defibrillation based on decreased intraventricular filling pressures and thereby terminate the electrical disturbance. In one example, the output from processor 60B is configured to communicate with an implanted cardiac device (such as a pacemaker or a defibrillator) to prepare or initiate therapy to terminate the arrhythmia. An implanted pacemaker can initiate the anti-tachycardia pacing or an implanted defibrillator can deliver an anti-arrhythmia shock to terminate the underlying electrical disturbance.

Various factors can affect the hemodynamic performance of the heart including the rate of chamber filling and chamber emptying when connected to an assist device. Over compensation of either may lead to further compromised performance of the heart. In view of the electro-mechanical coupling, the heart may become more susceptible to initiation of unintended cardiac arrhythmias. In one example, processor 60B provides an output corresponding to a detected pressure or a valve state (open or closed).

One example is directed to modulating contractility or pulsatility. Both the cardiac assist device 30D (FIG. 2) and heart 80A (FIG. 1A) contribute to the power with which blood is pumped to the body. A hemodynamic signal from sensor 40H (FIG. 2), such as an applanation tonometry sensor, can be digitally analyzed and fedback to the cardiac assist device 30D in order to maintain a balance between the blood flow provided by cardiac assist device 30D and native blood flow produced by heart 80A. An example of the present subject matter can be configured to discern the workload imposed on heart 80A and adjust the cardiac assist device 30D operational parameter to achieve a particular performance.

In one example, reverse remodeling can be induced in order to promote size reduction in an enlarged heart.

One example of the present subject matter is configured to detect, control, or modulate thrombosis or obstruction formation. As noted in other portions of this document, valve state can be detected and an operational parameter of the cardiac assist device 30D can be modulated to control flow and minimize stasis, and there by reduce or eliminate the incidence of thrombus. In addition, an occlusion in blood flow within a conduit, a connection, or a graft can be evaluated or identified based on a characteristic of the spectral content. In one example, an occlusion in blood flow can be evaluated or identified by comparison of the spectral content with a reference. The occlusion can be a blockage, an obstruction, or other irregularity and can occur in an intake side or an output side of a cardiac assist device.

One example is configured to detect failure or malfunction of cardiac assist device 30D. For example, a detected anomaly in spectral content corresponding to an uncommanded change in electrical current draw can be associated with a failure of the cardiac assist device 30D. One example can facilitate diagnoses of various types of pump malfunctions, such as intermittent power spikes from wire fatigue and damage. Tracking of applanation tonometry signals over time can assist in diagnosing pump malfunction problems.

One example is configured to measure or sense a signature of ventricular dys-synchrony and generate an output to adjust device parameters to improve ventricular synchrony and to improve cardiac output.

One example is configured to monitor hemodynamic information and adjust an operational parameter of cardiac assist device 30D in order to promote cardiac resynchronization. For example, processor 60B can generate an output configured to maintain an adequate heart rate such as may arise in conjunction with changes in patient physical activity.

One example is configured to diagnose cardiac remodeling. For example, over a period, processor 60B calculates and stores (in memory 220), a score corresponding to risk stratification. This can include generating an assessment of both short-term variability in the hemodynamic information (such as applanation tonometry signals) and long-term variability in the hemodynamic information. One example is configured to induce reverse remodeling based on an output generated by processor 60B and provided to cardiac assist device 30D.

### Part 4

### Additional

In one example, the pump speed of cardiac assist device 30D is an independent variable and dependent variables (which can be derived from the pump speed) include the pump flow, the pulse index (pulsatile index), and pump power consumption (in units of power, such as watts). With increasing pump speed, the pump flow will rise, the power consumption will rise, and the pulsatile index will drop. The pulsatile index approaches a near continuous flow and the relative amount of native pulse contribution to the blood flow is reduced. In essence, an increased pump speed will offload the pumping burden on the heart and increase the loading on the pump of the cardiac assist device. Some effects from increased pump speed include an increase in blood flow, a reduced power in the power spectrum, and a shift in the frequency components to lower frequencies.

An example of the present subject matter includes a system to establish a proper pump speed using a non-invasive sensor. In addition, the pump speed can be modulated with greater frequency and provide real-time (or near real-time modulation) in order to more closely match the cardiac assist device performance with the activities and health of the patient.

Ratiometric analysis of the spectral content can be used to diagnose various heart conditions of a patient, diagnose a cardiac device, and to control an operational parameter of the cardiac assist device. For example, the amplitude of a fundamental frequency spectral component can be used as a denominator in a ratio of two spectral components. The output generated by processor 60B can be calculated based on a product of at least two spectral components, based on a summation of at least two spectral components, or based on a difference of at least two spectral components. Other calculations having terms derived from data in the spectral content can also be used to determine the output.

Processor 60B can be configured to generate an output based on a calculated value or based on a comparison of a calculated value with a reference. The reference for comparison can be a numeric value or a frequency spectrum. The reference can be stored in memory 220. The reference can be a calculated or measured value derived from the specific patient or from a population.

The output can be generated by processor 60B using various calculations based on values derived from the spectral content. The various calculations can be used alone or in combination with at least one other calculation in generating an output. In one example, the output can be derived directly from an amplitude or frequency component associated with the fundamental frequency component or a harmonic. In one example, a ratio can be calculated using values derived from the spectral content. In one example, a mean value (such as an RMS value) can be calculated based on the spectral content.

In addition to calculating a quotient, a product, a difference and a sum, other calculations can be used to generate an output. For example, a differential or an integral based on the spectral content can be used to generate an output. Furthermore, other computational methods can be utilized to generate an output. Examples include generating an output by conducting principal component analysis (PCA), or independent component analysis (ICA), or singular value decomposition (SVD), wavelet analysis, windowing, or other methods.

In one example, the frequency domain representation is generated without first normalizing the amplitude of the time domain signal. In this example, later analysis is conducted based on frequency components and other aspects not dependent on signal amplitude.

An example of the present subject matter provides a real-time method, based in part on pulse wave analysis techniques, to adjust or optimize the performance of a cardiac assist device. Physiological signals of interest (such as blood pressure or heart rate) can be acquired from a patient using applanation tonometry or a cardiac assist device, for example, and can be processed using digital signal processing techniques and used for device optimization.

An example of the present subject matter is configured to acquire at least one real-time physiological signals of interest from a patient implanted with a ventricular assist device, analyze the signal using digital signal processing techniques, and provide optimized device parameters back to the assist device after making a clinical diagnosis.

The list provided below includes non-limiting examples of selected systems and methods, according to the present subject matter.

In Example 1, a system can include a sensor and a processor. The sensor can be configured to generate hemodynamic information for a patient. The processor can be configured to execute instructions to calculate spectral content using the hemodynamic information and configured to generate an output signal based on the calculated spectral content. The calculated spectral content can include a fundamental component and at least one harmonic component. The calculated spectral content can correspond to at least one of amplitude and frequency. The output signal can correspond to a state of the patient or correspond to an operational parameter of a cardiac assist device associated with the patient.

In Example 2, the system of Example 1 optionally configured such that the sensor includes an arterial tonometer.

In Example 3, the system of any one or a combination of Examples 1 or 2 wherein the sensor is optionally coupled to the cardiac assist device.

In Example 4, the system of any one or any combination of Examples 1-3 wherein the processor is optionally configured to normalize the hemodynamic information.

In Example 5, the system of any one or any combination of Examples 1-4 wherein the processor is optionally configured to calculate a power spectrum of the spectral content.

In Example 6, the system of any one or any combination of Examples 1-5 wherein the processor is optionally configured to compare the spectral content with a reference.

In Example 7, the system of any one or any combination of Examples 1-6 wherein the processor is optionally configured to generate a statistical profile of the spectral content.

In Example 8, the system of claim 7 wherein the processor is optionally configured to compare the statistical profile with a reference.

In Example 9, the system of any one or any combination of Examples 1-8 wherein the processor is optionally configured to determine a state of a cardiac valve.

In Example 10, the system of any one or any combination of Examples 1-9 wherein the processor is optionally configured to determine a pump speed.

In Example 11, the system of any one or any combination of Examples 1-10 wherein the processor is optionally configured to evaluate the patient state for at least one of CAD, ischemic heart disease, hypertension, CHF, left-sided heart failure, right-sided heart failure, bi-ventricular heart failure, SHF, DHF, systolic dysfunction, diastolic dysfunction, acute decompensation, AI, AR, AS, MR, MI, MS, and a cardiac valvular disease.

In Example 12, the system of any one or any combination of Examples 1-11 wherein the processor is optionally configured to perform a math operation using the spectral content, the math operation including multiplication, division, addition, or subtraction.

In Example 13, the system of any one or any combination of Examples 1-12 wherein the processor is optionally configured to calculate a mean of a power spectrum corresponding to the spectral content and further wherein the processor is configured to compare the mean with a reference.

In Example 14, the system of any one or any combination of Examples 1-12 wherein the processor is optionally configured to calculate a root mean square (RMS) value of a power spectrum corresponding to the spectral content and further wherein the processor is configured to compare the RMS value with a reference.

In Example 15, the system of any one or any combination of Examples 1-14 wherein the processor is optionally configured to adjust the operational parameter to correlate the calculated spectral content with a reference spectral content.

In Example 16, the system of any one or any combination of Examples 1-15 wherein the processor is optionally configured to communicate a notification signal based on the output signal.

In Example 17, the system of any one or any combination of Examples 1-16 wherein the processor is optionally configured to store the output signal in a memory.

In Example 18, the system of any one or any combination of Examples 1-17 optionally including a kinematic sensor coupled to the processor, the kinematic sensor configured to generate a kinematic signal corresponding to at least one of patient position and patient acceleration, and further wherein the output signal is determined based on the kinematic signal.

In Example 19, a method comprises receiving hemodynamic information for a patient, processing the hemodynamic information and generating an output signal. The method includes using a processor to process the hemodynamic information to calculate spectral content including a fundamental component and at least one harmonic component. The calculated spectral content corresponds to at least one of amplitude and frequency. The method includes generating an output signal based on the calculated spectral content. The output signal corresponds to a state of the patient or corresponds to an operational parameter of a cardiac assist device associated with the patient.

In Example 20, the method of Example 19 wherein receiving the hemodynamic information optionally includes receiving arterial tonometry information.

In Example 21, the method of any one or any combination of Examples 19 or 20 wherein receiving the hemodynamic information optionally includes receiving information from a non-invasive sensor.

In Example 22, the method of any one or any combination of Examples 19-21 wherein processing the hemodynamic information optionally includes normalizing.

In Example 23, the method of any one or any combination of Examples 19-22 wherein processing the hemodynamic information optionally includes performing a Fourier transform.

In Example 24, the method of any one or any combination of Examples 19-23 wherein generating the output signal optionally includes comparing the spectral content with a reference.

In Example 25, the method of any one or any combination of Examples 19-24 wherein comparing the spectral content with a reference optionally includes evaluating for an occlusion or thrombus formation, determining a state of a cardiac valve of the patient, diagnose internal bleeding, or identifying an arrhythmia.

In Example 26, the method of any one or any combination of Examples 19-25 wherein generating the output signal optionally includes generating a statistical profile of the spectral content.

In Example 27, the method of Example 26 optionally including comparing the statistical profile with a reference.

In Example 28, the method of any one or any combination of Examples 19-27 wherein generating the output signal optionally includes evaluating for an occlusion or thrombus formation, determining a state of a cardiac valve of the patient, diagnose internal bleeding, or identifying an arrhythmia.

In Example 29, the method of any one or any combination of Examples 19-28 wherein generating the output signal optionally includes determining a pump speed.

In Example 30, the method of any one or any combination of Examples 19-29 wherein generating the output signal optionally includes evaluating the patient state for at least one of CAD, ischemic heart disease, hypertension, CHF, left-sided heart failure, right-sided heart failure, bi-ventricular heart failure, SHF, DHF, systolic dysfunction, diastolic dysfunction, acute decompensation, AI, AR, AS, MR, MI, MS, and a cardiac valvular disease.

In Example 31, the method of any one or any combination of Examples 19-30 wherein generating the output signal optionally includes performing a math operation using the spectral content, the math operation including multiplication, division, addition, or subtraction.

In Example 32, the method of any one or any combination of Examples 19-31 optionally including calculating a mean of a power spectrum corresponding to the spectral content and wherein generating the output signal includes comparing the mean with a reference.

In Example 33, the method of Example 32 wherein calculating the mean optionally includes calculating a root mean square (RMS) value.

In Example 34, the method of any one or any combination of Examples 19-33 optionally including adjusting the operational parameter to correlate the calculated spectral content with a reference spectral content.

In Example 35, the method of any one or any combination of Examples 19-34 optionally including communicating a notification signal based on the output signal.

In Example 36, the method of any one or any combination of Examples 19-35 optionally including storing the output signal in a memory.

In Example 37, the method of any one or any combination of Examples 19-36 optionally including receiving a kinematic signal from a kinematic sensor, the kinematic signal corresponding to at least one of patient position and patient acceleration, and further wherein the output signal is determined based on the kinematic signal.

The above Detailed Description includes references to the accompanying drawings, which form a part of the Detailed Description. The drawings show, by way of illustration, specific embodiments in which the present optimization of cardiac assist devices using pulse wave techniques can be practiced. These embodiments are also referred to herein as “examples.”

The above Detailed Description and attached appendices are intended to be illustrative, and not restrictive. For example, the above- or attach-described examples (or one or more elements thereof) can be used in combination with each other. Other embodiments can be used, such as by one of ordinary skill in the art upon reviewing the above or attached description. Also, various features or elements can be grouped together to streamline the disclosure. This should not be interpreted as intending that an unclaimed disclosed feature is essential to any claim. Rather, inventive subject matter can lie in less than all features of a particular disclosed embodiment. Thus, the following claims and attached appendices are hereby incorporated into the Detailed Description, with each claim or embodiment standing on its own as a separate embodiment. The scope of the invention should be determined with reference to the appended claims, along with the full scope of equivalents to which such claims are entitled.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

In this document, the terms “a” or “an” are used to include one or more than one, independent of any other instances or usages of “at least one” or “one or more.” In this document, the term “or” is used to refer to a non-exclusive or, such that “A or B” includes “A but not B,” “B but not A,” and “A and B,” unless otherwise indicated.

In the appended claims, the terms “including” and “in which” are used as the plain-English equivalents of the respective terms “comprising” and “wherein.” The terms “including” and “comprising” are open-ended, that is, a system, kit, or method that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

1. A system comprising:
a sensor configured to generate hemodynamic information for a patient; and
a processor configured to execute instructions to calculate spectral content using the hemodynamic information and configured to generate an output signal based on the calculated spectral content, the calculated spectral content including a fundamental component and at least one harmonic component, the calculated spectral content corresponding to at least one of amplitude and frequency and wherein the output signal corresponds to a state of the patient or corresponds to an operational parameter of a cardiac assist device associated with the patient.

2. The system of claim 1 wherein the sensor includes an arterial tonometer.

3. The system of claim 1 wherein the sensor is coupled to the cardiac assist device.

4. The system of claim 1 wherein the processor is configured to normalize the hemodynamic information.

5. The system of claim 1 wherein the processor is configured to calculate a power spectrum of the spectral content.

6. The system of claim 1 wherein the processor is configured to compare the spectral content with a reference.

7. The system of claim 1 wherein the processor is configured to generate a statistical profile of the spectral content.

8. The system of claim 7 further wherein the processor is configured to compare the statistical profile with a reference.

9. The system of claim 1 wherein the processor is configured to determine a state of a cardiac valve.

10. The system of claim 1 wherein the processor is configured to determine a pump speed.

11. The system of claim 1 wherein the processor is configured to evaluate the patient state for at least one of coronary artery disease (CAD), ischemic heart disease, hypertension, congestive heart failure (CHF), left-sided heart failure, right-sided heart failure, bi-ventricular heart failure, systolic heart failure (SHF), diastolic heart failure (DHF), systolic dysfunction, diastolic dysfunction, acute decompensation, aortic insufficiency (AI), aortic regurgitation (AR), aortic stenosis (AS), mitral regurgitation (MR), mitral insufficiency (MI), mitral stenosis (MS), and a cardiac valvular disease.

12. The system of claim 1 wherein the processor is configured to perform a math operation using the spectral content, the math operation including multiplication, division, addition, or subtraction.

13. The system of claim 1 wherein the processor is configured to calculate a mean of a power spectrum corresponding to the spectral content and further wherein the processor is configured to compare the mean with a reference.

14. The system of claim 1 wherein the processor is configured to calculate a root mean square (RMS) value of a power spectrum corresponding to the spectral content and further wherein the processor is configured to compare the RMS value with a reference.

15. The system of claim 1 wherein the processor is configured to adjust the operational parameter to correlate the calculated spectral content with a reference spectral content.

16. The system of claim 1 wherein the processor is configured to communicate a notification signal based on the output signal.

17. The system of claim 1 wherein the processor is configured to store the output signal in a memory.

18. The system of claim 1 further including a kinematic sensor coupled to the processor, the kinematic sensor configured to generate a kinematic signal corresponding to at least one of patient position and patient acceleration, and further wherein the output signal is determined based on the kinematic signal.

19. A method comprising:
receiving hemodynamic information for a patient;
processing, using a processor, the hemodynamic information to calculate spectral content including a fundamental component and at least one harmonic component, the calculated spectral content corresponding to at least one of amplitude and frequency; and
generating an output signal based on the calculated spectral content, wherein the output signal corresponds to a state of the patient or corresponds to an operational parameter of a cardiac assist device associated with the patient.

20. The method of claim 19 wherein receiving the hemodynamic information includes receiving arterial tonometry information.

21. The method of claim 19 wherein receiving the hemodynamic information includes receiving information from a non-invasive sensor.

22. The method of claim 19 wherein processing the hemodynamic information includes normalizing.

23. The method of claim 19 wherein processing the hemodynamic information includes performing a Fourier transform.

24. The method of claim 19 wherein generating the output signal including comparing the spectral content with a reference.

25. The method of claim 24 wherein comparing the spectral content with the reference includes evaluating for an occlusion or thrombus formation, determining a state of a cardiac valve of the patient, diagnose internal bleeding, or identifying an arrhythmia.

26. The method of claim 19 wherein generating the output signal includes generating a statistical profile of the spectral content.

27. The method of claim 26 further including comparing the statistical profile with a reference.

28. The method of claim 19 wherein generating the output signal includes evaluating for an occlusion or thrombus formation, determining a state of a cardiac valve of the patient, diagnose internal bleeding, or identifying an arrhythmia.

29. The method of claim 19 wherein generating the output signal includes determining a pump speed.

30. The method of claim 19 wherein generating the output signal includes evaluating the patient state for at least one of coronary artery disease (CAD), ischemic heart disease, hypertension, congestive heart failure (CHF), left-sided heart failure, right-sided heart failure, bi-ventricular heart failure, systolic heart failure (SHF), diastolic heart failure (DHF), systolic dysfunction, diastolic dysfunction, acute decompensation, aortic insufficiency (AI), aortic regurgitation (AR), aortic stenosis (AS), mitral regurgitation (MR), mitral insufficiency (MI), mitral stenosis (MS), and a cardiac valvular disease.

31. The method of claim 19 wherein generating the output signal includes performing a math operation using the spectral content, the math operation including multiplication, division, addition, or subtraction.

32. The method of claim 19 further including calculating a mean of a power spectrum corresponding to the spectral content and wherein generating the output signal includes comparing the mean with a reference.

33. The method of claim 32 wherein calculating the mean includes calculating a root mean square (RMS) value.

34. The method of claim 19 further including adjusting the operational parameter to correlate the calculated spectral content with a reference spectral content.

35. The method of claim 19 further including communicating a notification signal based on the output signal.

36. The method of claim 19 further including storing the output signal in a memory.

37. The method of claim 19 further including receiving a kinematic signal from a kinematic sensor, the kinematic signal corresponding to at least one of patient position and patient acceleration, and further wherein the output signal is determined based on the kinematic signal.
